(19) United States
(12) Patent Application Publication
(10) Pub. No.: US 2017/0145470 A1
(43) Pub. Date: May 25, 2017

(54) SPECIMEN TRANSFER APPARATUS AND METHOD

(71) Applicant: Fahad Mohammedsaleh ALBUKHARI, Oxford, MS (US)

(72) Inventor: Fahad Mohammedsaleh ALBUKHARI, Oxford, MS (US)

(21) Appl. No.: 15/350,541

(22) Filed: Nov. 14, 2016

Related U.S. Application Data
(60) Provisional application No. 62/259,292, filed on Nov. 24, 2015.

(51) Int. Cl. C12Q 1/24; G01N 35/00; G01N 1/08

(52) U.S. Cl. CPC C12Q 1/24; G01N 1/08; G01N 35/00871; G01N 2001/085; G01N 2035/00188

(57) ABSTRACT

A specimen transfer apparatus includes control processing circuitry and a specimen support surface including a support base. The apparatus includes a lift mechanism to drive the specimen support surface and a drive mechanism to drive a drive rod rotatably connected to a drive motor for rotation about an axis. The apparatus includes a die cutter rotatably connected to the drive rod via support arms connected to the die cutter. The die cutter rotates from a first position to a second position about the axis of the drive rod. The specimen support surface includes a sensor array disposed in the specimen support surface. The processing circuitry is configured to detect a position of a first plate and a second plate via the sensor array. The lift and drive mechanisms are electrically connected to the processing circuitry. The processing circuitry is further configured to separately control the lift and drive mechanisms.

### CROSS-REFERENCE TO RELATED APPLICATIONS

This application is based on, and claims the benefit of priority to, provisional application No. 62/259,292, filed Nov. 24, 2015, the entire contents of which are incorporated herein by reference.

### GRANT OF NON-EXCLUSIVE RIGHT

This application was prepared with financial support from the Saudi Arabian Cultural Mission, and in consideration therefore the present inventor(s) has granted The Kingdom of Saudi Arabia a non-exclusive right to practice the present invention.

### BACKGROUND

Field of the Disclosure

This disclosure relates to the identification of filamentous fungi, molds, and yeasts, and specifically to a specimen transfer apparatus and method for the same.

Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Many species of fungi cause disease in plant, animal and human. Identification of these fungi is important in establishing the etiology of the disease and in prescribing a course of treatment.

Identification of fungi is accomplished by isolating and growing these organisms on appropriate solid culture media and observing their macroscopic and microscopic appearance. Colonial morphology may be of little value in the identification of filamentous fungi due to natural variation among isolates and variation which is culture medium dependent. The definitive identification of filamentous fungi is based on the characteristic morphology of the arrangement of spores and fruiting bodies. The production of these structures is encouraged by growing these organisms on specific types of culture media. To microscopically visualize the arrangement of spores and fruiting bodies a slide culture may be prepared. This is accomplished by filling a petri dish with an appropriate nutrient medium such as Sabouraud's dextrose agar (SabDex), corn meal agar, or potato dextrose agar (PDA). From this petri dish of agar medium a small block of agar is aseptically cut, removed and placed on a sterile slide. The agar block is inoculated on the surface with the fungus to be identified. After inoculation a sterile cover slip is placed over the top surface of the agar block. The inoculated slide-agar block is placed in an empty sterile petri dish which contains a supporting mechanism for the slide.

There are many variations on this method, all requiring elaborate preparations. One is to dip a coverslip into liquid agar, allowing it to solidify and placing it on a sterile slide in a petri dish.

All current methods for growing fungi in slide cultures are based on the principle of incubation in a moist chamber. The state of the art methods require the use and construction of many components, all of which must be sterilized individually. It is laborious, time consuming, and does not allow for standardization. Contamination of the components by other organisms is frequently encountered.

When transferring fungal cultures aseptically to maintain a pure plate or petri dish while performing experimentation, it is desirable that the fungal culture remain pure and not contaminated with other organisms or even other species. Thus, aseptic techniques are used to maintain a sterile environment to keep the cultures pure. The tools that have been used to transfer fungal cultures are usually a scalpel, forceps, a cork borer (or a sterile test tube), and a spatula. For the sterile environment, alcohol and an alcohol lamp may also be used.

Aseptic technique is a fundamental and important laboratory skill in the field of microbiology. Microbiologists use aseptic technique for a variety of procedures such as transferring cultures, inoculating media, isolation of pure cultures, and for performing microbiological tests. Proper aseptic technique prevents contamination of cultures from foreign bacteria inherent in the environment. For example, airborne microorganisms (including fungi), microbes picked up from the researcher's body, the lab bench-top or other surfaces, microbes found in dust, as well as microbes found on unsterilized glassware and equipment, etc. may potentially contaminate cultures, thus interfering with the lab results. Using proper aseptic technique can greatly minimize or even eliminate the risk of contamination. In addition, aseptic technique is of utmost importance to maintain pure stock cultures while transferring cultures to new media. Aseptic technique is also essential for isolation of a single species of microorganism from a mixed culture to obtain a pure culture. Furthermore, proper aseptic technique prevents microbes used in the laboratory from accidentally being released into the environment and/or infecting people working in the laboratory. This is especially relevant when pathogens are being handled.

Therefore, the mechanism or procedure for fungal culture transfer to maintain a pure plate begins with a user washing his/her hands with soap and then sterilizing their hands with the alcohol. Next, the user puts on gloves which are then treated with alcohol as well. Now a cut is made inside the agar plate from the edge of the agar of block shape (piece of fungal culture on agar) with the scalpel after dipping the scalpel in the alcohol and flaming the scalpel for sterilization until glowing red over the alcohol lamp. Afterwards, the scalpel may be cooled down in the excess agar which is no longer needed or not being used for the agar block. At the same time, the user must flip the agar block 180 degrees (upside down) and then place it on a new pure plate or petri dish for the fungal culture to grow in the new pure plate. The forceps may be used to flip the block after the agar is cut via the scalpel.

The above describes a process of the transfer of fungal cultures from one pure plate to another pure plate using a scalpel and forceps. However, there is another method to transfer fungal cultures to a pure petri dish, which is using the cork borer pore and spatula. The procedure of transferring fungal cultures to maintain a pure plate begins with dipping the cork borer in alcohol, and flame sterilizing the scalpel until glowing red over the alcohol lamp. Next, the scalpel is cooled by dipping it into a practice block of agar that is no longer needed. In some instances a sterile test tube is used to cut the agar instead of using the cork borer. The sterilized scalpel is then used to flip the block upside down, and replace on the new pure petri dish for growing. Sometimes, forceps is used to flip the block instead of the scalpel. To prevent the agar plate from dehydrating, which may eventually kill the fungi, the plate is wrapped in, for example, a paraffin film, such as PARAFILM. Finally, the streaked agar plate is incubated either overnight in a 37° C. incubator or at room temperature, for 48 hours.

### SUMMARY

Embodiments include a specimen transfer apparatus including control processing circuitry and a specimen support surface including a support base. The apparatus also includes a lift mechanism to drive the specimen support surface and a drive mechanism to drive a drive rod rotatably connected to a drive motor for rotation about an axis. The apparatus further includes a die cutter rotatably connected to the drive rod via support arms integrally connected to the die cutter. The die cutter rotates from a first position to a second position about the axis of the drive rod. The specimen support surface includes a sensor array disposed in the specimen support surface. The control processing circuitry is configured to detect a position of a first pure plate and a position of a second pure plate via the sensor array. The lift mechanism and the drive mechanism are electrically connected to the control processing circuitry. The control processing circuitry is further configured to separately control the lift mechanism and drive mechanism.

Embodiments also include a method for specimen transfer including providing an indication that a first pure plate on a specimen support surface is disposed at a first pre-set location and providing an indication that a second pure plate on the specimen support surface is disposed at a second pre-set location. The method also includes determining whether the first pure plate and the second pure plate are aligned on the specimen support surface at a first pre-set location and a second pre-set location, respectively. The method further includes raising the specimen support surface to a location proximal a die cutter. The method also includes cutting a specimen from the first pure plate via the die cutter. The method further includes gripping the specimen via the die cutter and rotating the die cutter 180 degrees from the first pre-set location to the second pre-set location. The method also includes releasing and transferring the cut specimen from the die cutter to the second pure plate.

Embodiments further include a specimen transfer apparatus including means for indicating a first pure plate on a specimen support surface is disposed at a first pre-set location and means for indicating a second pure plate on the specimen support surface is disposed at a second pre-set location. The apparatus also includes means for determining whether the first pure plate and the second pure plate are aligned on the specimen support surface at a first pre-set location and a second pre-set location, respectively. The apparatus further includes means for raising the specimen support surface to a location proximal a die cutter. The apparatus also includes means for cutting a specimen from the first pure plate via the die cutter. The apparatus further includes means for gripping the specimen via the die cutter and rotating the die cutter 180 degrees from the first pre-set location to the second pre-set location. The apparatus also includes means for releasing and transferring the cut specimen from the die cutter to the second pure plate.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is an illustrative view of a specimen transfer apparatus according to certain embodiments of the disclosure;

FIG. 2 is an illustrative view of a die cutter attached to the specimen transfer apparatus of FIG. 1 according to certain embodiments of the disclosure.

FIG. 3 is an isolated perspective view of the die cutter according to certain embodiments of the disclosure.

FIG. 4A is an illustrative view of a first specimen plate showing a removed sample according to certain embodiments of the disclosure.

FIG. 4B is an illustrative view of a second specimen plate including the removed sample from the first specimen plate according to certain embodiments of the disclosure.

FIG. 5 is a block diagram of a controller for the die cutter apparatus according to certain embodiments of the disclosure.

FIG. 6 is a block diagram of a sensor array for the die cutter apparatus according to certain embodiments of the disclosure.

FIG. 7 is a block diagram of a drive mechanism for the die cutter apparatus according to certain embodiments of the disclosure.

FIG. 8 is a flowchart of a method for specimen transfer according to certain embodiments of the disclosure, and

FIG. 9 is a schematic diagram of the controller of FIG. 5 according to certain embodiments of the disclosure.

FIG. 10 illustrates a second die cutter according to certain embodiments of the disclosure.

FIG. 11 illustrates a first handheld specimen transfer apparatus in first position according to certain embodiments of the disclosure.

FIG. 12 illustrates working of the first handheld specimen transfer apparatus according to certain embodiments of the disclosure.

FIG. 13 illustrates a second handheld specimen transfer apparatus according to certain embodiments of the disclosure.

FIG. 14 illustrates working of the second handheld specimen transfer apparatus according to certain embodiments of the disclosure.

FIG. 15A illustrates a first transfer device implementing the first transfer apparatus in FIG. 11 according to certain embodiments of the disclosure.

FIG. 15B illustrates a second transfer device implementing the second transfer apparatus in FIG. 13 according to certain embodiments of the disclosure.

FIG. 16A illustrates a third specimen transfer device according to certain embodiments of the disclosure.

FIG. 16B is a perspective view of the third specimen transfer device according to certain embodiments of the disclosure.

FIG. 17A is a front view of a fourth specimen transfer device in a first position according to certain embodiments of the disclosure.

FIG. 17B is a perspective view of the fourth specimen transfer device in the first position according to certain embodiments of the disclosure.

FIG. 18A is a front view of the fourth specimen transfer device in a second position according to certain embodiments of the disclosure.

FIG. 18B is a perspective view of the fourth specimen transfer device in the second position according to certain embodiments of the disclosure.

FIG. 19A is a front view of the fourth specimen transfer device in a third position according to certain embodiments of the disclosure.

FIG. 19B is a perspective view of the fourth specimen transfer device in the third position according to certain embodiments of the disclosure.

FIG. 20 is a front view of the fifth specimen transfer device in a third position according to certain embodiments of the disclosure.

FIG. 21A is a front view of a fifth cutter of the fifth specimen transfer device according to certain embodiments of the disclosure.

FIG. 21B is a top view of the fifth cutter of the fifth specimen transfer device according to certain embodiments of the disclosure.

FIG. 21C is a side view of the fifth cutter of the fifth specimen transfer device according to certain embodiments of the disclosure.

FIG. 22A is a front view of a plunger of the fifth specimen transfer device according to certain embodiments of the disclosure.

FIG. 22B is a side view of the plunger of the fifth specimen transfer device according to certain embodiments of the disclosure.

### DETAILED DESCRIPTION OF THE EMBODIMENTS

In the drawings, like reference numerals designate identical or corresponding parts throughout the several views.

In certain embodiments, the present disclosure provides an apparatus that can transport an agar block from a first petri dish or pure plate into a new second petri dish or pure plate. The apparatus may help to save time in the method of isolation (aseptic) and inoculation. The apparatus may have similar characteristics of a pipette or chemical dropper in that the apparatus may hold and then transfer a specimen from one position to another.

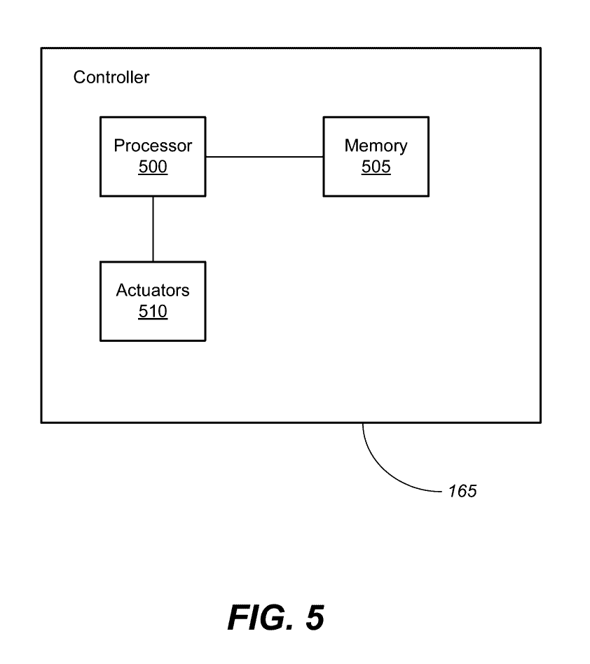

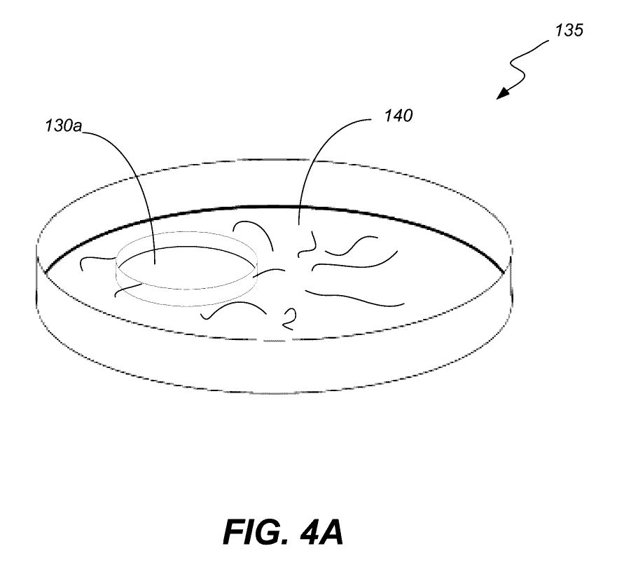

The disclosed apparatus may operate in a way similar to a pattern cutting die. The apparatus may have a disposable cutter that can cut the sample/specimen from a first pure plate and lift the specimen inside the cutter, and then flip the sample/specimen 180 degrees and deposit the specimen into a second pure plate for later processing (incubation). Next, the apparatus may simply lay or drop the sample/specimen onto a different (second) pure plate with the original surface of the sample/specimen facing the surface of the second pure plate. To clarify, the surface of the sample/specimen in the first plate is what is visualized (see FIG. 4A at 140).

In some embodiments, the disclosed apparatus may cut the sample/specimen and flip it 180 degrees, and at this point the surface that was visualized (proximal a top surface) would be facing downward. Then, the apparatus may drop the sample/specimen into a second dish, where the surface that was visualized on the first dish will be facing the top surface of the second dish. In other words, the exposed surface of the sample/specimen at this point (flipped) will be facing the top surface of the second dish.

In certain embodiments, the apparatus may include a cutter and a holder of the sample/specimen that is disposable to simplify the clean/aseptic technique. One advantage of the disclosed apparatus may be that lab workers will save time and energy on sterilizing the tools necessary for such actions.

In certain embodiments, the present disclosure includes an apparatus configured to transfer fungal culture blocks or the like from a first agar petri dish to a second (pure plate) agar petri dish by making a die cut inside the first agar petri dish from the edge of the agar block and flipping while holding the cut agar block 180 degrees and then releasing the cut agar block onto the second agar petri dish. Thus, the visualized surface (top) of the agar block will be facing downward (upside down) in the second agar petri dish or plate. Therefore, the surface that was visualized in the first agar petri dish now faces the top surface of the second agar petri dish.

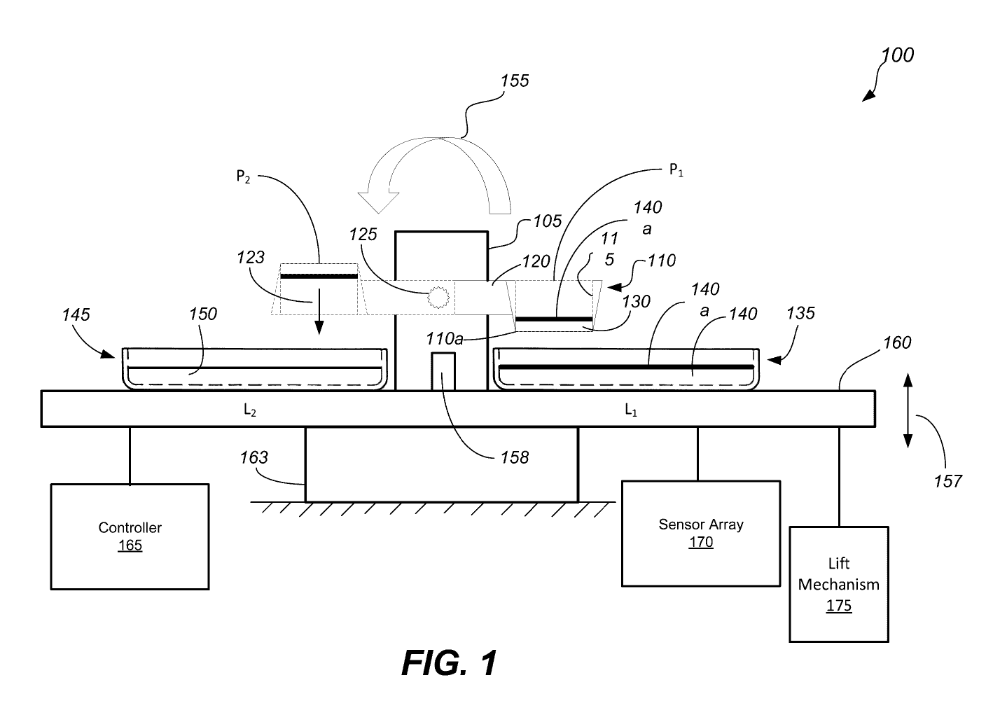

FIG. 1 is an illustrative view of a specimen transfer apparatus 100 according to certain embodiments of the disclosure. In FIG. 1, apparatus 100 may include a drive mechanism 105, a die cutter 110, a support surface portion 160, a base portion 163, a controller 165, a sensor array 170, and a lift mechanism 175.

Drive mechanism 105 may include a splined rotatable drive rod 125 having an axis configured to rotate in a direction 155 moving die cutter 110 from a first position P₁ to a second position P₂ (shown in phantom) by 180° about the axis. That is, the die cutter 110 occupies either of position P₁ or P₂ by rotating 180° about an axis of drive rod 125.

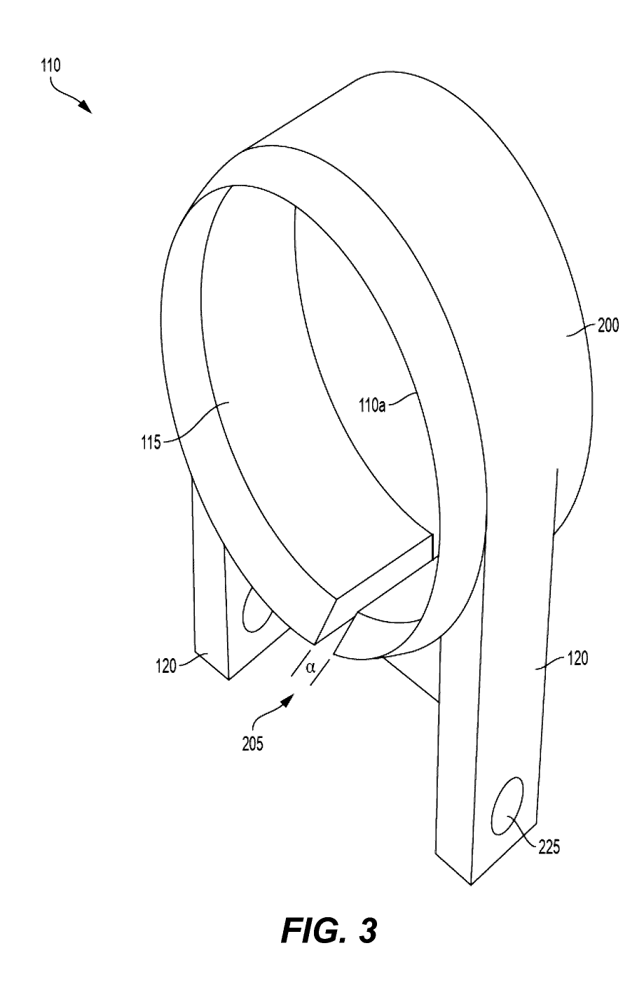

Die cutter 110 may include a hollow interior wall portion 115 configured to accommodate and grip a sample 130 obtained from sample specimen 140. In some embodiments, interior wall portion 115 of die cutter 110 may be coated with TEFLON or the like to allow for the sample 130 to more easily release from the die cutter 110 to its destination on the agar 150 of second plate 145 (see FIGS. 1 and 4B at 130). In certain embodiments, the interior wall portion 115 of die cutter 110 may include a smooth surface or a textured or rough frictional surface to hold the die cut agar sample 130. Die cutter 110 also may include a cutting portion 110a as a sharpened edge disposed along a circumferential portion of the die cutter 110 (see FIG. 3). Die cutter 110 may further include integral support arms 120 attached at a distal end to rod 125 for rotation. Die cutter 110 is configured to cut, grip, move, and release sample 130 during use. Die cutter 110 is also configured to be easily attached and removed from splined rotatable rod 125. Die cutter 110 can be provided as a disposable pre-sterilized part supplied with apparatus 100. In some embodiments, die cutter 110 is made of a rigid yet flexible material such as sterile plastic, surgical steel, or the like. When made of metal, such as surgical steel, die cutter 110 may be flame and/or alcohol sterilized as discussed above prior to being attached to rod 125.

Support surface portion 160 may be movably connected to lift mechanism 175 for movement in a direction 157 along a spaced groove 158. Support surface portion 160 may be configured to support a first pure plate 135 and a second pure plate 145 each disposed proximal die cutter 110. First pure plate 135 may include an agar having a specimen 140 having an active surface 140a, such as a fungal culture. Second pure plate 145 may include an agar 150, such as potato dextrose agar (PDA).

In some embodiments, the top surface of the sample 130 from the first plate 135 is what is visualized at a moment. The apparatus 100 may be configured to cut the sample 130 and flip the specimen 180° with respect to base portion 163, and at that point the surface 140a that was visualized would be facing down. Then, in the embodiment the apparatus 100 will drop the sample 130 into a second dish 145, where the surface 140a that was visualized on the first dish 135 will be facing the surface of the second dish 145 (facing down).

In apparatus 100, lift mechanism 175 may be configured to move support surface portion 160 toward die cutter 110 being in first position P₁ or while die cutter 110 may be in an upright neutral position. The die cutter 110 then contacts with the specimen 140 to cut and hold a portion of the specimen 140 as the sample 130 having active surface 140a. Upon cutting and gripping sample 130, die cutter 110 is configured to rotate 180° from position P₁ above the first pure plate 135 about axis 125 toward the second pure plate 145 at position P₂. During this rotation to position P₂, sample 130 is flipped over with the active surface 140a now directly facing agar 150. Next at position P₂, apparatus 100 is configured to release sample 130 in a direction 123 to impact agar 150, the release mechanism discussed below. Now, a user may proceed with further testing or incubation of plate 145 as by adding a cover seal, or the like to plate 145.

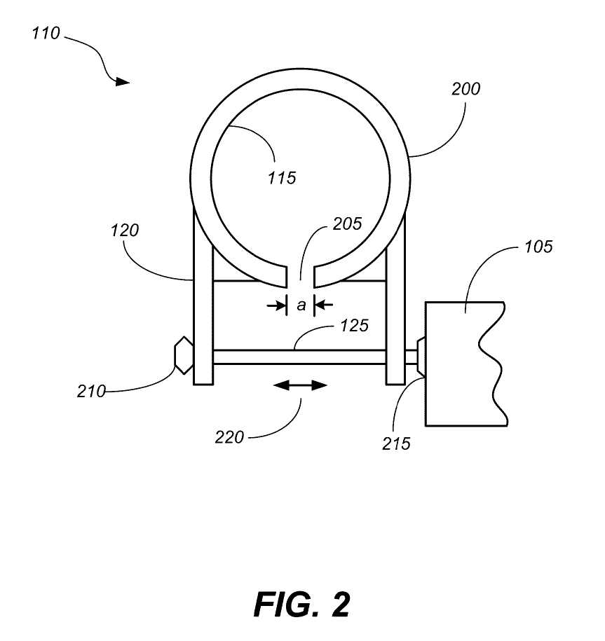

FIG. 2 is an illustrative view of a die cutter 110 attached to the specimen transfer apparatus 100 of FIG. 1 according to certain embodiments of the disclosure. In FIG. 2, die cutter 110 may include hollow interior wall portion 115 and integral support arms 120 rotatably attached to rod 125. In some embodiments, interior wall portion 115 may be configured to include either a smooth, low frictional surface or a frictional textured surface. Die cutter 110 also includes an exterior wall portion 200 and a spaced gap portion 205. Spaced gap portion 205 may be configured to be spaced apart a distance a, for example, 1 to 3 millimeters (mm). In some embodiments, die cutter 110 may be generally cylindrical in form and shape, and could be formed without the gap 205.

In some embodiments, die cutter 110 may be a disposable part of apparatus 100. Further, die cutter 110 may be configured as a cylindrical loop including exterior wall portion 200 having a closed end and a diametrically opposed opened spaced gap portion 205 at a. The gap portion 205 at a is configured to flex inward (close) and outward (open) when pressure or a force is applied to integral support arms 120 to cause die cutter 110 to either grip or release a die cut sample 130, such as a fungal sample of agar or other microorganism.

In some embodiments, die cutter 110 may be rotatably mounted to rod 125 via a removable fastener 210 disposed at a distal end of rod 125. In certain embodiments, fastener 210 may be configured as a snap-fit or catch, while in other embodiments, fastener 210 may be configured as a locking nut or the like. A proximal end of rod 125 may include a washer or spacer 215 with rod 125 mounted into drive mechanism 105 for both rotation movement (see FIG. 1 at 155) and for translational movement in a direction 220 to open or close gap portion 205. In other words, the translational movement in direction 220 provides tension to die cutter 110 at support arms 120 when gripping sample 130 by decreasing distance a of gap portion 205 during a cut and later releasing the tension placed upon gap portion 205 via support arms 120, by increasing distance a to release sample 130 allowing it to fall and impact upon agar 150 in plate 145. Gap portion 205 provides a spring-like action to occur in die cutter 110 when under tension and then released. In other embodiments, rod 125 may be configured to eject from drive mechanism 105 so a user may easily remove a used die cutter 110 for quick replacement procedure by inserting a new die cutter 110 on rod 125 and reinserting rod 125 into drive mechanism 105 for subsequent die cutting.

Die cutter 110 may also be configured to be pivotal and include at least one of mounting support arms 120 integrally attached to its exterior wall portion 200. In some embodiments, die cutter 110 is configured as a single-use cutter to be disposable after every use to minimize the need for tedious sterilization techniques as discussed above.

FIG. 3 is an isolated perspective view of the die cutter 110 according to certain embodiments of the disclosure. In FIG. 3, die cutter 110 includes cutting portion 110a (a sharpened edge), hollow interior wall portion 115, integral support arms 120 including a rod connector portions 225, exterior wall portion 200, and gap portion 205. Rod connector portions 225 are configured to allow the insertion of rod 125. Rod connector portions 225 may be configured to be interlocking with splined rotatable rod 125.

In some embodiments, cutting portion 110a may include a cutting teeth configuration. In other embodiments, cutting portion 110a may be disposed on opposite ends of exterior wall portion 200 along a circumferential edge.

In certain embodiments, the die cutter 110 may be configured to include dual-sided cutting edges (beveled) to provide a clean or neat cut of the agar sample 130. In other words, die cutter 110 may be configured as a double-sided cutter to provide a long lasting cutting tool which can be re-used and sterilized until both cutting portions 110a are dulled at which time the die cutter 110 may be disposed of.

FIG. 4A is an illustrative view of a first specimen plate 135 showing a removed sample space 130a according to certain embodiments of the disclosure. In FIG. 4A, plate 135 may be a petri dish or the like including specimen 140, such as a fungal culture. Removed sample space 130a coincides with sample 130 shown in FIG. 1 which was cut and gripped by die cutter 110. Apparatus 100 may be configured to provide an indicator to a user via sensor array 170 that plate 135 is in a proper position on support surface portion 160 to receive a die to cut specimen 140 for further processing. In some embodiments, sensor array 170 is disposed in support surface portion 160.

FIG. 4B is an illustrative view of a second specimen plate 145 including the removed sample 130 from the first specimen plate 135 according to certain embodiments of the disclosure. In FIG. 4B, plate 145 may be a petri dish or the like including agar 150, such as PDA to feed sample 130 upon contact with active surface 140a of sample 130. Apparatus 100 may be configured to provide an indicator to a user via sensor array 170 that plate 145 is in a proper position on support surface portion 160 to receive the cut specimen 140 for further processing.

FIG. 5 is a block diagram of a controller 165 for the die cutter apparatus 100 according to certain embodiments of the disclosure. In FIG. 5, controller 165 may include at least one processor 500, at least one memory 505, and actuators 510. Controller 165 may be configured, via the processor 500, to trigger, via actuators 510, the movement of die cutter 110 including the gripping and release action of the die cutter 110 on sample 130. Memory 505 may be configured to store certain types of data on particular specimens or different sized pure plates used in apparatus 100. In other words, some specimens or different sized pure plates may require a different sized die cutter 110 or positioning of the plates on support surface portion 160 may change. Actuators 510 may include activation and deactivation of apparatus 100 and/or the triggering of features in the drive mechanism 105 discussed below. For example, in certain embodiments, actuators 510 may include a plurality of actuators. For example, some embodiments may include three actuators 510 which include a first actuator configured to grab/release agar sample 130, a second actuator configured to install/dispose of die cutter 110, and a third actuator configured to rotate die cutter 110 about axis 125 by 180°. The first, second, and third actuators may be configured to operate via instructions from processor 500 and/or by a user manually triggering them in the instance when any automated controls fail or a programming code glitch occurs.

In operation, for example, a user may activate via buttons, mouse, or other input device the actuators 510, and activate the second actuator to release an old die cutter and to install a new die cutter. After the die cutting operation has plunged the die cutter 110 into the agar specimen 140, activate the first actuator to grip the agar sample 130 inside the die cutter 110 as described above. Next, activate the third actuator to rotate the agar sample 130 over the second petri dish/plate 145 via die cutter 110 with agar sample 130 being gripped inside interior wall portion 115 of die cutter 110. Then, activate the first actuator again to trigger the release of agar sample 130 onto plate 145. Finally, activate the second actuator to dispose of the used die cutter 110.

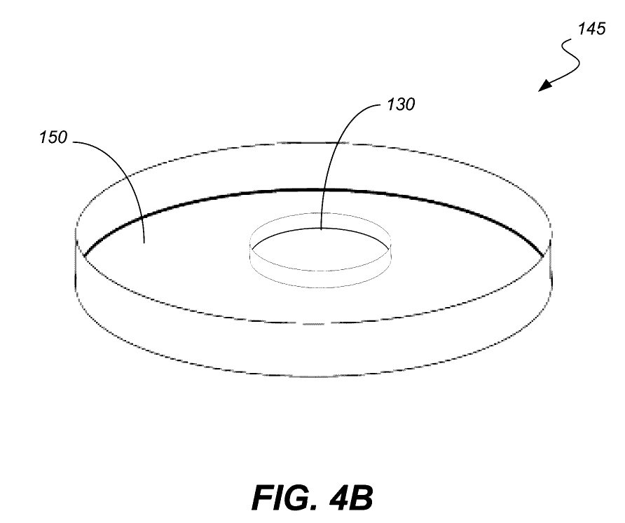

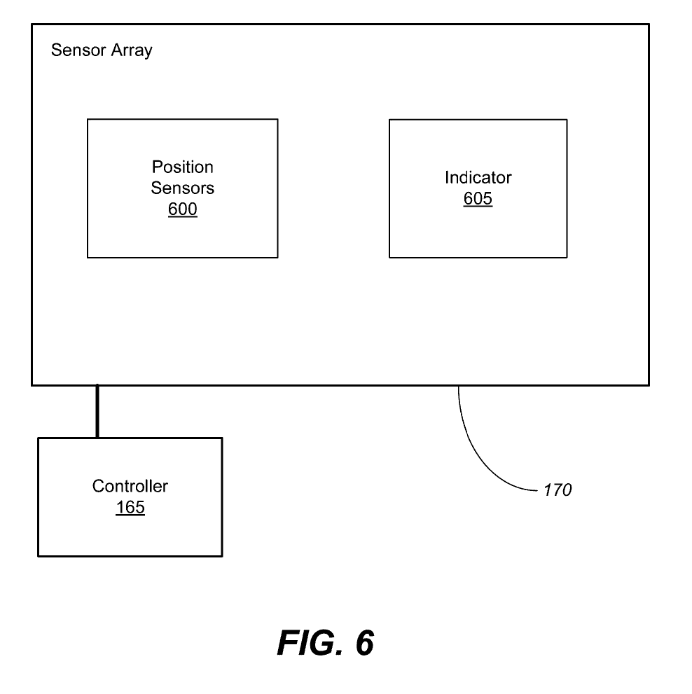

FIG. 6 is a block diagram of a sensor array 170 for the die cutter apparatus 100 according to certain embodiments of the disclosure. In FIG. 6, sensor array 170 may include position sensors 600 configured to align first pure plate 135 and second pure plate 145 into position (L₁, L₂) to cut sample 130 from specimen 140 in plate 135 and to transfer sample 130 to plate 145. Position sensors 600 may include, for example, a potentiometric position sensor a capacitance position sensor, a fiber-optic position sensor, an optical position sensor, a Eddy-current position sensor, a linear voltage differential transformers, a proximity sensor, a magnetostrictive linear position sensor, and the like, and combinations of the same.

Further, sensor array 170 may be configured to indicate via an indicator 605 to a user, the relative positions of plate 135 and plate 145 with respect to each other and/or the die cutter 110 so that the user can properly align the plates 135, 145 prior to cutting and transferring sample 130 from plate 135 to plate 145 for further processing or handling. These relative positions may be a first pre-set location L₁ for plate 135 and a second pre-set location L₂ for plate 145 on support surface portion 160 of apparatus 100. Indicator 605 may include an audible, graphic, or visual indication that plate 135 and plate 145 are in proper position or alignment with die cutter 110. Sensor array 170 may be disposed within support surface portion 160 proximal die cutter 110. Controller 165 may be electrically connected to sensor array 170 to receive feedback indicating that plate 135 and plate 145 are in proper position to proceed with a die cut.

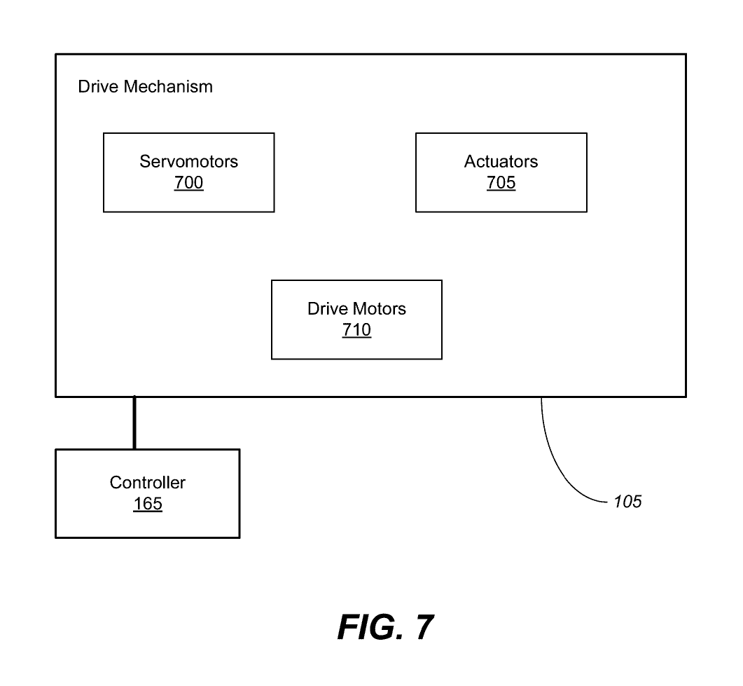

FIG. 7 is a block diagram of a drive mechanism 105 for the die cutter apparatus 100 according to certain embodiments of the disclosure. In FIG. 7, drive mechanism 105 may include servomotors 700, actuators 705, and drive motors 710.

Servomotors 700 may be configured to move rod 125 translationally from a first grip position (in which distance a of gap 205 is reduced) to a second release position (in which distance a of gap 205 is increased) toward and away from drive mechanism 105 in direction 220. Actuators 705 may include activation and deactivation control of drive mechanism 105. In some embodiments, actuators 705 may include an ejector mechanism to easily eject or remove the die cutter 110 for disposal once used and contaminated by contact with specimen 140 during use. Drive motors 710 may be configured to rotate rod 125 from a cutting and gripping position from plate 135 to a 180° releasing position for sample 130 to plate 145. Controller 165 may be electrically connected to drive mechanism 105 to signal activation of the die cutter 110 and/or the ejection of die cutter 110 for disposal when used or contaminated by contact with specimen 140 during use.

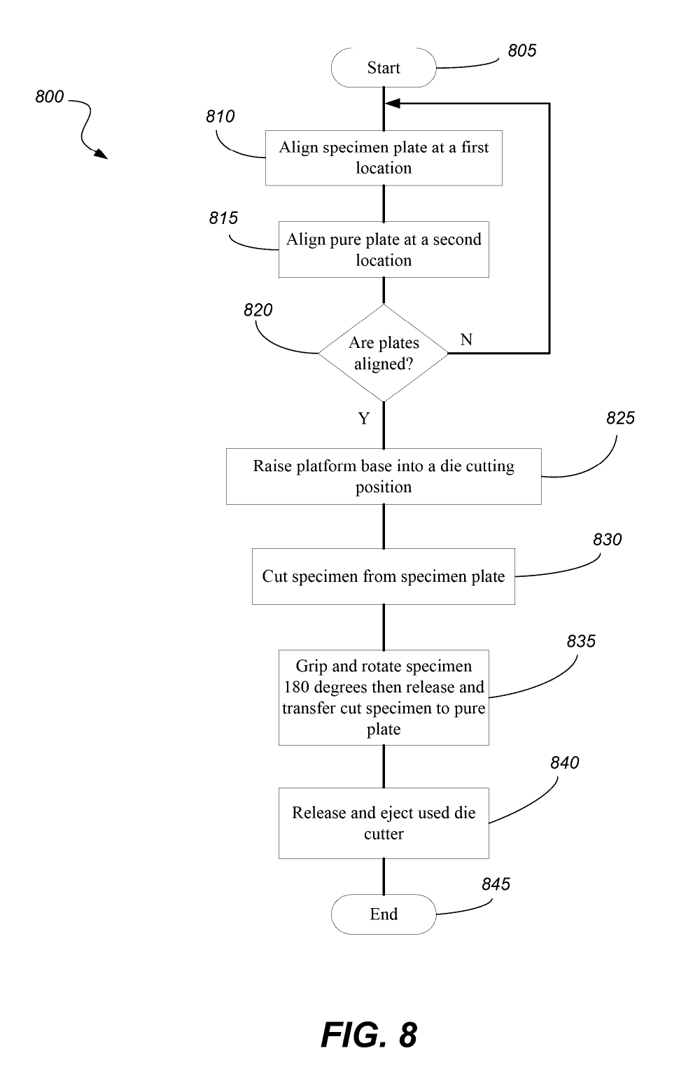

FIG. 8 is a flowchart of a method 800 for specimen transfer according to certain embodiments of the disclosure. In FIG. 8, method 800 starts at 805. At 810, a first specimen plate 135 is aligned by a user at a first pre-set location L₁ on apparatus 100 at a first position indicated by indicator 605. At 815, a second pure plate 145 is aligned by a user at a second pre-set location L₂ on apparatus 100 at a second position indicated by indicator 605. At 820, sensor array 170 confirms whether both plate 135 and plate 145 are properly aligned via position sensors 600, such as a proximity sensor. At 825, support surface portion 160 is raised into a die cutting position by lift mechanism 175. At 830, die cutter 110 contacts the specimen 140 and sample 130 is cut from specimen 140 on the plate 135. At 835, sample 130 is gripped and rotated 180 degrees via die cutter 110, then sample 130 is released and transferred effectively from plate 135 to plate 145. At 840, the used die cutter 110 is released and ejected from apparatus 100 via controller 165 (this operation is optional if the die cutter 110 is reusable). At 845, method 800 ends and apparatus 100 may be deactivated.

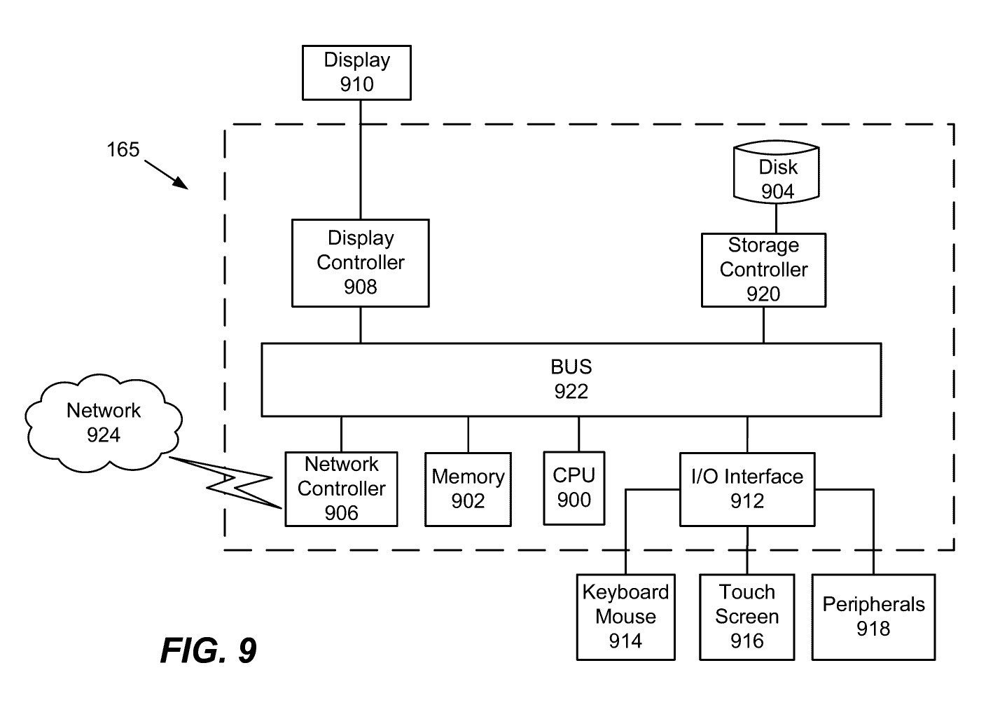

FIG. 9 is a schematic diagram of the controller 165 of FIG. 5 according to certain embodiments of the disclosure. In FIG. 9, a hardware description of the controller 165 according to exemplary embodiments is described. In FIG. 9, the controller 165 includes a CPU 900 which performs the processes described above/below. The process data and instructions may be stored in memory 902. These processes and instructions may also be stored on a storage medium disk 904 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the controller 165 communicates, such as a server or computer.

Further, the claimed advancements may be provided as a utility application, background daemon, or component of an operating system, or combination thereof, executing in conjunction with CPU 900 and an operating system such as Microsoft Windows®, UNIX®, Solaris®, LINUX®, Apple® MAC-OS and other systems known to those skilled in the art.

The hardware elements in order to achieve the controller 165 may be realized by various circuitry elements, known to those skilled in the art. For example, CPU 900 may be a Xeon® or Core® processor from Intel® of America or an Opteron® processor from AMD® of America, or may be other processor types that would be recognized by one of ordinary skill in the art. Alternatively, the CPU 900 may be implemented on an FPGA, ASIC, PLD or using discrete logic circuits, as one of ordinary skill in the art would recognize. Further, CPU 900 may be implemented as multiple processors cooperatively working in parallel to perform the instructions of the inventive processes described above.

The controller 165 in FIG. 9 also includes a network controller 906, such as an Intel® Ethernet PRO® network interface card from Intel® Corporation of America, for interfacing with network 924. As can be appreciated, the network 924 can be a public network, such as the Internet, or a private network such as an LAN or WAN network, or any combination thereof and can also include PSTN or ISDN sub-networks. The network 924 can also be wired, such as an Ethernet network, or can be wireless such as a cellular network including EDGE, 3G and 4G wireless cellular systems. The wireless network can also be WiFi, Bluetooth, or any other wireless form of communication that is known.

In certain embodiments, apparatus 100 may be controlled and operated remotely via network 924 by a user equipment, such as a smartphone, tablet computer, laptop computer or the like.

The controller 165 further includes a display controller 908, such as a NVIDIA® GeForce GTX® or Quadro® graphics adaptor from NVIDIA® Corporation of America for interfacing with display 910, such as a Hewlett Packard® HPL2445w LCD monitor. A general purpose I/O interface 912 interfaces with a keyboard and/or mouse 914 as well as a touch screen panel 916 on or separate from display 910. General purpose I/O interface also connects to a variety of peripherals 918 including printers and scanners, such as an OfficeJet® or DeskJet® from Hewlett Packard®.

The general purpose storage controller 920 connects the storage medium disk 904 with communication bus 922, which may be an ISA, EISA, VESA, PCI, or similar, for interconnecting all of the components of the controller 165. A description of the general features and functionality of the display 910, keyboard and/or mouse 914, as well as the display controller 908, storage controller 920, network controller 906, and general purpose I/O interface 912 is omitted herein for brevity as these features are known.

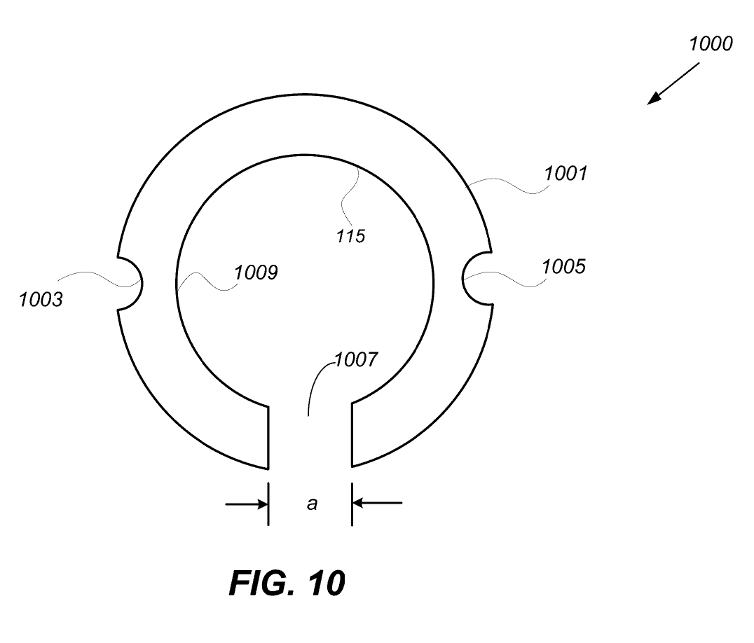

FIG. 10 illustrates a second die cutter according to certain embodiments of the disclosure. The second die cutter 1000 can be any device having a ring-like circular structure that can cut the sample 130 from the first specimen plate 135, hold the sample 130 and release the sample 130 in the second specimen plate 145. The second die cutter 1000 can include a cutting edge 1009 (a sharpened edge similar to cutting portion 110a of the die cutter 110), a hollow interior wall portion 115 (shown in FIG. 3), an outer wall 1001 having indents 1003 and 1005, and a gap 1007. The indents 1003 and 1005 can be formed 180° apart in the outer wall 1001. The gap 1007 may be configured to be spaced apart a distance a, for example, 1 to 3 millimeters (mm). In some embodiments, the second die cutter 1000 may be generally cylindrical in form and shape, and could be formed without the gap 1007.

The cutting edge 1009 can have a cutting teeth configuration or a smooth sharp edge. In certain embodiments, the second die cutter 1000 may be configured to include dual-sided cutting edges (beveled), similar to the die cutter 110, to provide a clean or neat cut of the agar sample 130.

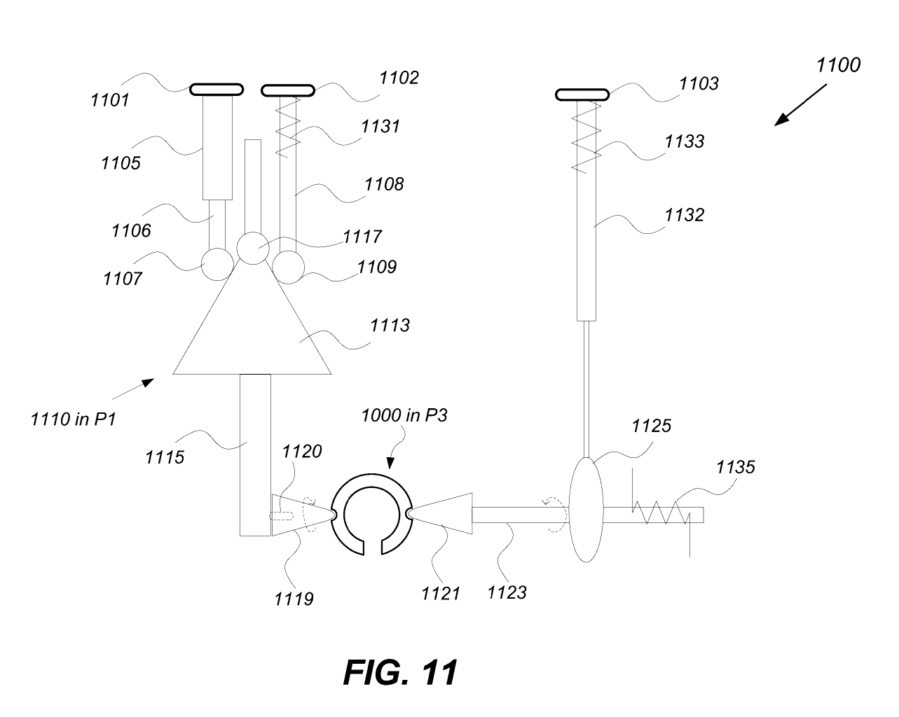

FIG. 11 illustrates a first handheld specimen transfer apparatus in first position according to certain embodiments of the disclosure. The first handheld specimen transfer apparatus 1100 can be any device that can hold a die cutter such as the die cutter 110 or 1000, apply pressure on the external wall, flip the die cutter by 180°, and release pressure on the die cutter.

The first handheld specimen transfer apparatus 1100 can include a hold button 1101, release button 1102, a rotate button 1103, and a pendulum 1110. The hold button 1101 can be connected to a hold shaft 1106 with a hold roller 1107. Similarly, the release button 1102 can be connected to a release shaft 1108 with a release roller 1109. The hold roller 1107 can move along the left slant edge of a conical portion 1113 of the pendulum 1110, while the release roller 1109 can move along the right slant edge of the conical portion 1113 of the pendulum 1110. The movement of the rollers exerts force on the conical portion 1113 causing the pendulum 1110 to swing freely about a pivot ball 1117. Further, springs 1131 and 1133 can be included to retract the release button 1102, and rotate button 1103 respectively.

The hold button 1101 and the hold shaft 1106 can be further configured to include a locking mechanism to lock the hold shaft 1106 in a depressed position when the hold button 1101 is pressed. The locking mechanism can be similar to a clicking mechanism 1105 used in a pen. The clicking mechanism is well known and not illustrated in detail for brevity. The clicking mechanism 1105 can include a cam profile on a hollow cylinder connected to the hold shaft 1106, a plunger and a spring. When the hold button 1101 is pressed, the hold shaft 1106 moves downwards while causing the cam profile to rotate and the plunger to lock the hold shaft 1106 in the depressed position.

The pendulum 1110 can be any device that pivots in clockwise or counter-clockwise direction at first end depending on the direction of an external force. The pendulum 1110 remains in a vertical position P1, when no force is applied. The external force can be applied by pressing the hold button 1101 causing the pendulum 1110 to rotate in counter-clockwise direction. Also, the external force can be applied by pressing the release button 1102 causing the pendulum 1110 to rotate in clockwise direction.

The pendulum 1110 can include the conical portion 1113, the pivot ball 1117, a pendulum shaft 1115. The apex of the conical portion 1113 can be pivotably connected to the pivot ball 1117, while the base of the conical portion 1113 can be connected to the pendulum shaft 1115. The pendulum shaft 1115 can be attached with a first hold cone 1119 at a free end or a second end using a pin 1120. The first hold cone 1119 can freely rotate about a pin 1120.

The rotate button 1103 can be any device that can turn a rotate shaft 1123 by approximately 180°, when pressed downwards and the rotate button 1103 can be retracted by a spring action of the spring 1133. The rotate button 1103 can be connected to a shaft 1132 coupled with a disk 1125 mounted on the rotate shaft 1123. Further, a return spring 1135 can be connected to the rotate shaft 1123 at one end and a second hold cone 1121 at the other end. When the rotate button 1103 is pressed downwards, the shaft 1132 turns the disk 1125 causing the rotate shaft 1123 to rotate by approximately 180°, while compressing the spring 113.

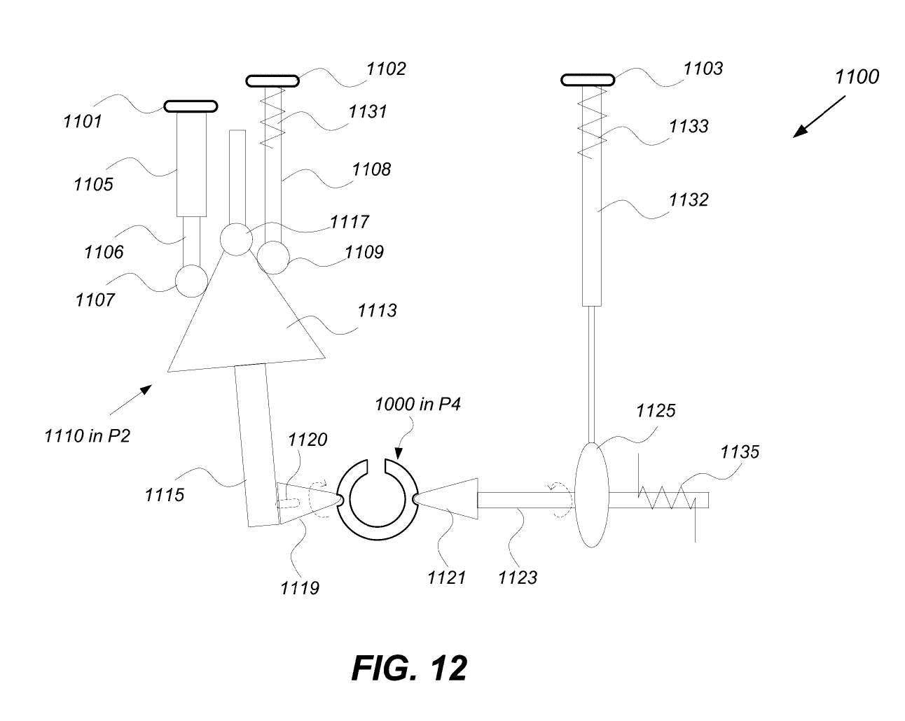

The second cutter 1000 can be placed between the first hold cone 1119 and the second hold cone 1121 in a third position P3. The tip of the hold cones 1119 and 1121 can rest in the indents 1003 and 1005 respectively to hold the second cutter 1000 in the first handheld transfer apparatus 1100. The pendulum 1110 can exert and release pressure on the second cutter 1000 using the hold button 1101 and the release button 1102 respectively. Further, the second cutter 1000 can be flipped by approximately 180° into a fourth position P4 (in FIG. 12) using the rotate button 1103.

FIG. 12 illustrates working of the first handheld specimen transfer apparatus according to certain embodiments of the disclosure. Initially, the release button 1102 is pressed and released to mount the second cutter 1000 is mounted in a third position P3. Then, the hold button 1101 is pressed and locked in depressed position by the clicking mechanism 1105 causing the pendulum 1110 to pivot and occupy position P2. In position P2, the pendulum 1110 exerts a force on the second cutter 1000 causing the second cutter 1000 to slightly compress. The first handheld transfer apparatus 1100 can be moved over the first specimen plate 135 and a sample 130a can be removed using the cutting edge 1009 of the second cutter 1000.

Once the sample 130a is removed, the first handheld transfer apparatus 1100 can be moved over the second specimen plate 145. The rotate button 1103 can be pressed to flip the second cutter 1000 holding the sample 130a upside down into the fourth position P4. While holding the rotate button 1103, the hold button 1101 is pressed to retract the hold button 1101 causing the pendulum to return to the vertical position P1 (in FIG. 11). As such, the pressure exerted on the second cutter 1000 is released and the sample 130a drops on the second specimen plate 145. Further, the release button 1102 can be pressed to release and replace the second cutter 1000.

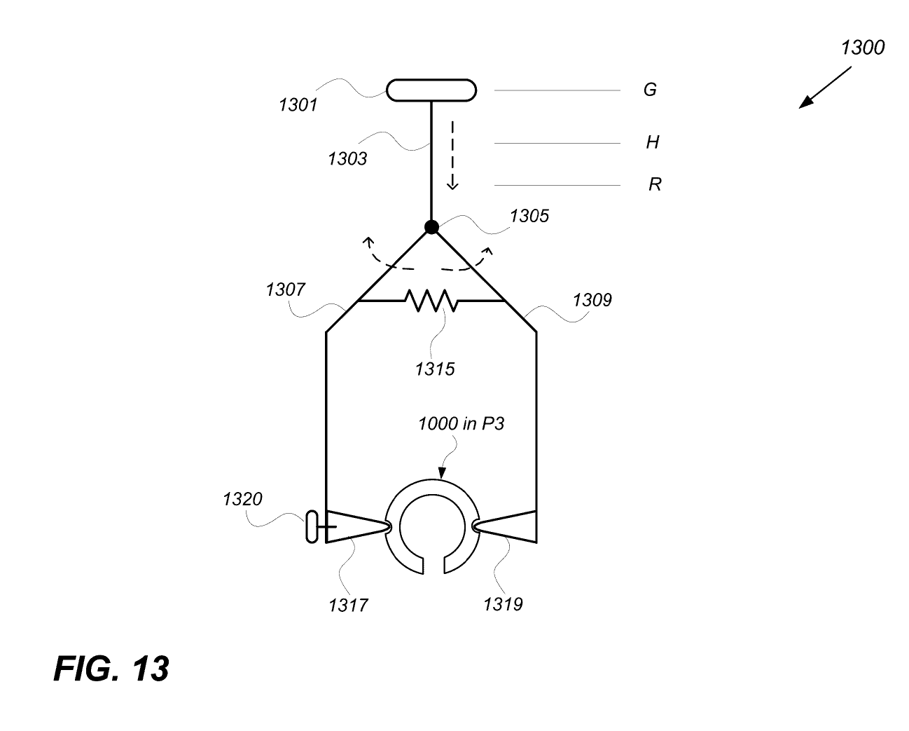

FIG. 13 illustrates a second handheld specimen transfer apparatus according to certain embodiments of the disclosure. The second handheld transfer apparatus 1300 can include a button 1301, a first link 1303, a pivot joint 1305, a second link 1307, a third link 1309, a first spring 1315, a rotator 1320, a third hold cone 1317 and a fourth hold cone 1319. The button 1301 can be configured to attain a grip position, a hold position or a release position by applying force on top of the button 1301. The grip position G can be attained when no force is applied on the button 1301. The hold position can be attained by pressing the button 1301 to a hold level H. The release position can be attained by pressing the button 1301 to a release level R. The distance between the grip level G and the hold level H or the release level R can depend on the size of the second die cutter 1100. The hold level H and the release level R can be pre-determined experimentally by varying the factors such as size of the die cutter, length of the links, and size of the hold cones.

The force applied on the button 1301 is transferred to the first link 1303 which pushes the pivot joint 1305 causing the second link 1307 and the third link 1309 to pivot. The second and the third links 1307 and 1309 can be hinged using the pivot joint 1305 and further connected by the first spring 1315.

The second link 1307 can be attached with the third hold cone 1317 which can be rotated approximately 1800 by the rotator 1320. The third link 1309 can be attached with the fourth hold cone 1319, which may rotate. The third cone 1317 and the fourth cone 1319 can hold the second cutter 1000 at indents 1003 and 1005.

The second link 1307 and the third link 1309 are pulled towards each other by the first spring 1315 causing the third cone 1317 and the fourth cone 1309 to exert pressure on the outer wall of the second cutter 1000. The second cutter 1000 can be released by pressing the button 1301 to the release level R.

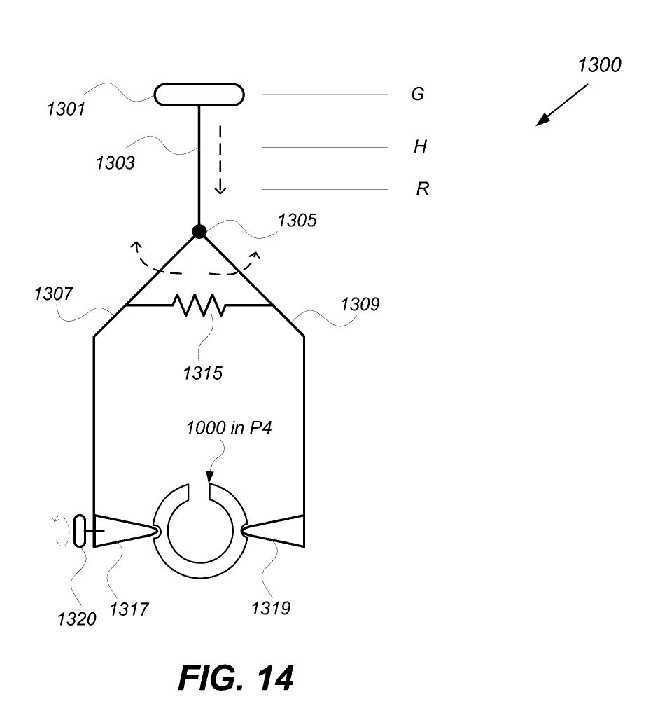

FIG. 14 illustrates working of the second handheld specimen transfer apparatus according to certain embodiments of the disclosure. Initially, the button 1301 can be pressed to release level R causing the third cone 1317 and the fourth cone 1319 to move away from each other. The cones can be aligned with the indents 1003 and 1005 of the second cutter 1000. The pressure on the button 1301 can be released causing the first spring 1315 to pull the third link 1307 and the fourth link 1309 towards each other and grip the second cutter 1000, while the button 1301 moves to the grip position G. The second handheld transfer apparatus 1300 can be moved over the first specimen plate 135 and a sample 130a can be removed using the cutting edge 1009 of the second cutter 1000.

Once the sample 130a is removed, the second handheld transfer apparatus 1300 can be moved over the second specimen plate 145. The rotator 1320 can be rotated to flip the second cutter 1000 holding the sample 130a upside down into the fourth position P4. Then, the button 1301 can be pressed to the hold level H causing the third and the fourth cones 1317 and 1319 to move away slightly releasing the pressure on the indents 1003 and 1005 of the second cutter 1000 while holding the second cutter 1000. The sample 130a drops on the second specimen plate 145. Further, the button 1301 can be pressed to release level R and the second cutter 1000 can be disposed and replaced.

In another embodiment, the first handheld specimen transfer apparatus 1100 and the second handheld specimen transfer apparatus 1300 can be used with the die cutter 110. Further, a present disclosure is not limited to die cutters 110 or 1000 and die cutter or a different shape and size can be adopted to use with the first and second specimen transfer apparatus 1100 and 1300 respectively.

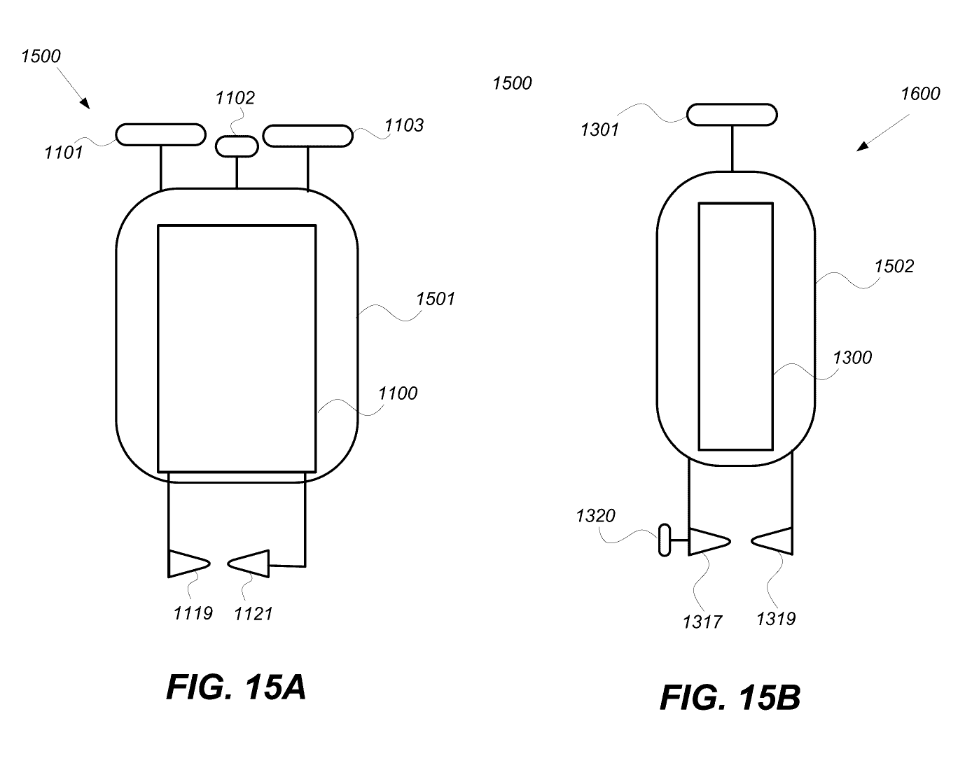

FIG. 15A illustrates a first transfer device implementing the first transfer apparatus in FIG. 11 according to certain embodiments of the disclosure. The first transfer device 1500 can be a handheld device including a first casing 1501 enclosing the first handheld specimen transfer apparatus 1100. The first casing 1501 encloses the components of the first handheld specimen 1100, while allowing the hold button 1101, the release button 1102, and the rotate button 1103 to project outside the casing. Also, the first and second hold cones 1119 and 1121 respectively can be allowed to project outside the casing.

FIG. 15B illustrates a second transfer device implementing the second transfer apparatus in FIG. 13 according to certain embodiments of the disclosure. Similar to the first transfer device 1500, a second transfer device 1600 can be a handheld device. The second transfer device 1600 can include a second casing 1502 enclosing the second handheld specimen transfer apparatus 1300. The second casing 1502 encloses the components of the second handheld specimen 1300, while allowing the button 1300 to be accessed outside the second casing 1502. Also, the third and fourth hold cones 1317 and 1319 respectively can be allowed to project outside the casing. The rotator 1320 can be accessed to rotate the third cone 1317.

In certain embodiments, a processing circuitry such as controller 165 can be included in the first transfer device 1500 and the second transfer device 1600 to control the rotation of the die cutter. For example, a motor configured to receive signal from the processing circuitry can be installed to drive the hold cones such as the first hold cones 1119 of the first transfer device 1500 or the third drive cone 1317 of the second transfer device 1600. Further, sensors can be installed and configured to send signals to the processing circuitry. For example, a position sensor can be installed in the first transfer device 1500 or the second transfer device 1600 to identify the position of the first specimen plate 135 and the second specimen plate 145. The position sensors can also be used to align the die cutter 1000 with the hold cones 1119, 1121, 1317, or 1319. The position sensor data can also be used to trigger indicators (such as red and green lights) for an incorrect alignment or correct alignment. Further, an rotation indicator confirming the rotation of the die cutter can also be installed. The rotation indication can be configured to receive signal from the processing circuitry based on the motor rotation or a position sensor.

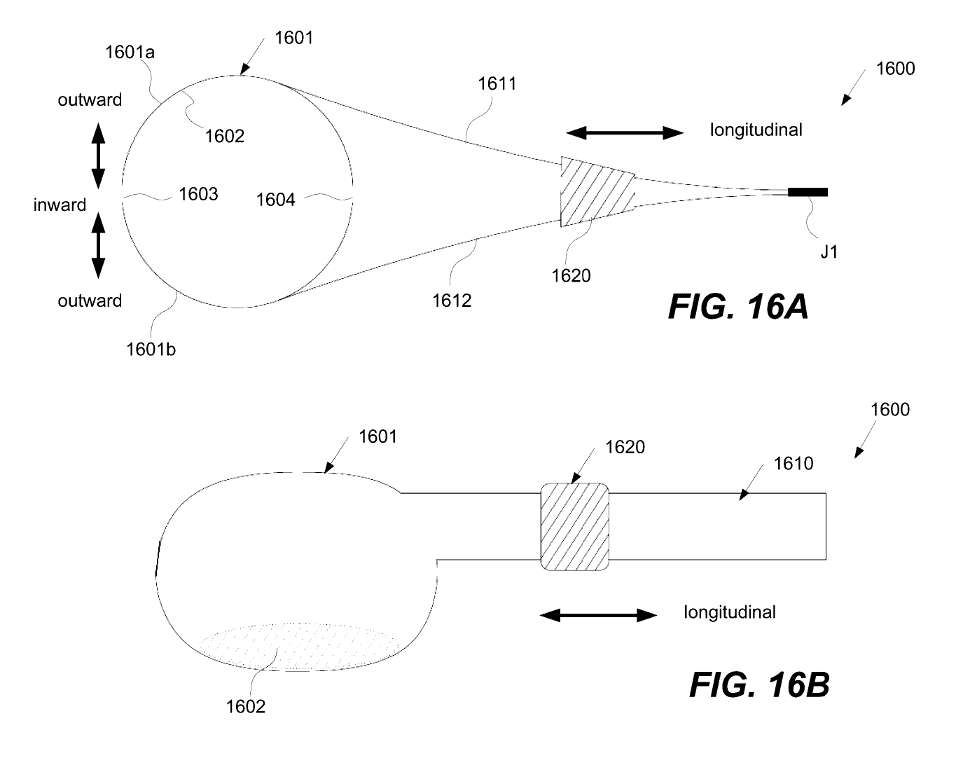

FIGS. 16A and 16B illustrate top view and a front view, respectively, of a third specimen transfer device 1600 according to certain embodiments of the disclosure. Referring to FIG. 16B, the third specimen transfer device 1600 (also referred as a third device 1600 hereinafter) includes a ring cutter 1601, a flexible housing 1610, and a shuttle 1620. The ring cutter 1601 is connected to the flexible housing 1610 and the shuttle 1620 slides back and forth along the flexible housing 1610 causing the ring cutter 1601 to compress or expand. The ring cutter 1601 can cut and hold a specimen (e.g., specimen 130a in FIG. 4A of the first specimen plate 135) for transferring the specimen to a different specimen plate (e.g., the second specimen plate 145 in FIG. 4B).

Referring to FIG. 16A, the ring cutter 1601 can have a ring like shape. The ring cutter 1601 includes a hollow interior wall 1602, a first slit 1603 and a second slit 1604. The hollow interior wall can be a smooth, low frictional surface or a frictional textured surface. The first slit 1603 and the second slit 1604 divides the ring cutter 1601 into two portions—a first cutter portion 1601a and a second cutter portion 1601b.

The ring cutter 1601 may be a disposable part of the device 1600. Further, the slits 1603 and 1604 can be configured to flex inward (close) and outward (open) when pressure or a force is applied to flexible housing 1610 to cause ring cutter 1601 to grip or to release the specimen 130a (in FIG. 4A) such as a fungal sample of agar or other microorganism.

The flexible housing 1610 includes two arms—a first arm 1611 and a second arm 1612. The first arm 1611 and the second arm 1612 are fixedly connected at an end J1 and can flex inward (close) and outward (open) when pressure or a force is applied.

The first arm 1611 is connected to the first cutter portion 1601a at a first end and the second end of the first arm 1611 is connected to a second end of the second arm 1612 at an end J1. The first end of the second arm 1612 is connected to the second cutter portion 1601b. The first arm 1611 and the second arm 1612 are held together by the shuttle 1620. The first arm 1611 and the second arm 1612 can be flexed inwards or outwards by moving the shuttle 1620 back and forth along the longitudinal direction. For example, when the shuttle 1620 slides towards the end J1, the arms 1611 and 1612 flex outwards. When the shuttle 1620 slides towards the ring cutter 1601 the arms 1611 and 1612 flex inwards.

The first arm 1611 and the second arm 1612 can be cylindrical rods or rectangular plates made of metal or plastic that allows the arms 1611 and 1612 to be flexible. The arms 1611 and 1612 can be joined at end J1 by welding, screw, rivet, adhesive, or other fastening methods. The first arm 1611 and the second arm 1612 can be connected to the ring cutter 1601 by removable joint such as a removable pin-joint to dispose or replace the ring cutter 1601.

The shuttle 1620 can be a hollow conical shaped piece with smooth inner surface to allow sliding along the arms 1611 and 1612 in a smooth manner. In another example, the shuttle 1620 can be a hollow cylinder, or a ring like component. The shuttle 1620 can be made of metal or plastic.

The operation of the third device 1600 is as follows. In a first position (i.e., an initial position), the shuttle 1620 is located at the end J1 and the slits 1603 and 1604 are open. The third device 1600 can be placed over the first specimen plate (not illustrated) and the ring cutter 1601 can be forced into the sample in the first specimen plate to cut a specimen. Then, the shuttle 1620 can be moved towards the ring cutter 1601 causing the ring cutter 1601 to clamp or grip the specimen. Finally, the third device 1600 can be moved over the second specimen plate and the shuttle 1620 can be moved to the end J1 causing the ring cutter 1601 to release the specimen to the second specimen plate.

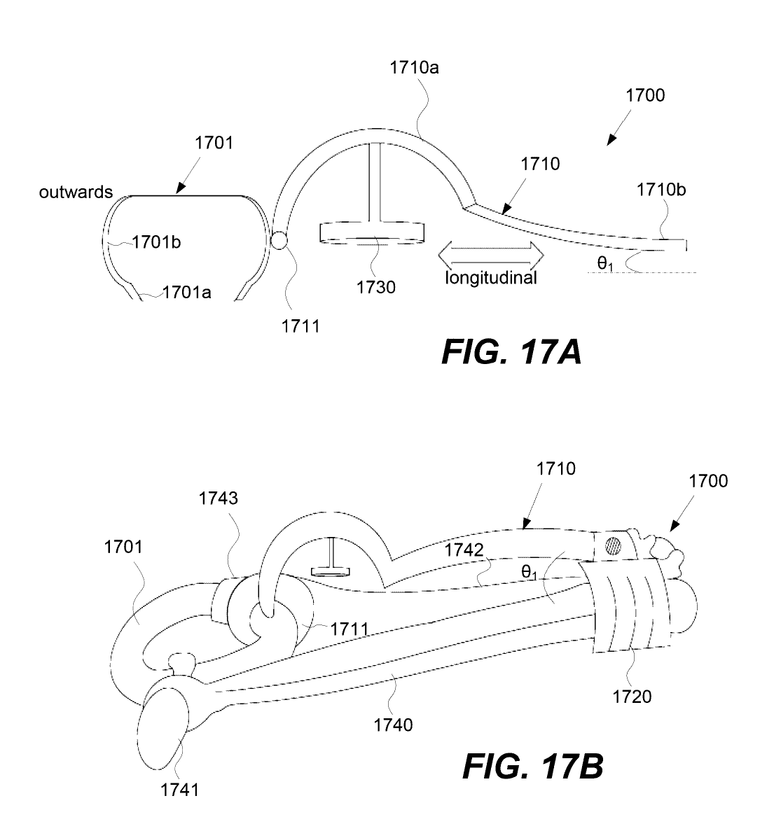

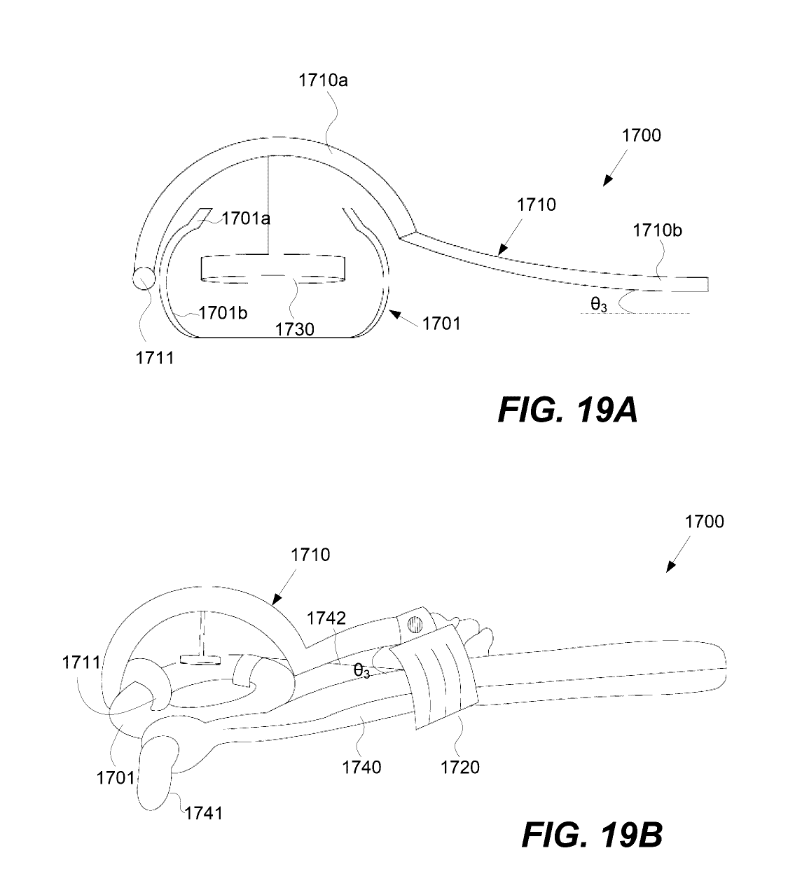

FIGS. 17A and 17B illustrate plan view and a perspective view, respectively, of a fourth specimen transfer device 1700 in a first position according to certain embodiments of the disclosure. The fourth specimen transfer device (also referred as a fourth device 1700 hereinafter) includes a second cutter 1701, a movable arm 1710, an arm compressor 1720, a plunger 1730, a first supporting arm 1740, and a second supporting arm 1742. The second cutter 1701 is rotatably connected to the first supporting arm 1740 and the second supporting arm 1742 by the first joint 1741 and the second joint 1743, respectively. The second cutter 1701 is also rotatably connected to the movable arm 1710 by a third joint 1711. The third joint 1711 acts as a pivot point about which the second cutter 1701 rotates from an initial position (in FIG. 17A) to an inverted position (in FIG. 19A). The third joint 1711 can be a ball joint while the first and the second joints 1741 and 1743 can be a simple hinge joint that allows the second cutter 1701 to rotate about the first supporting arm 1740 and the second supporting arm 1742.

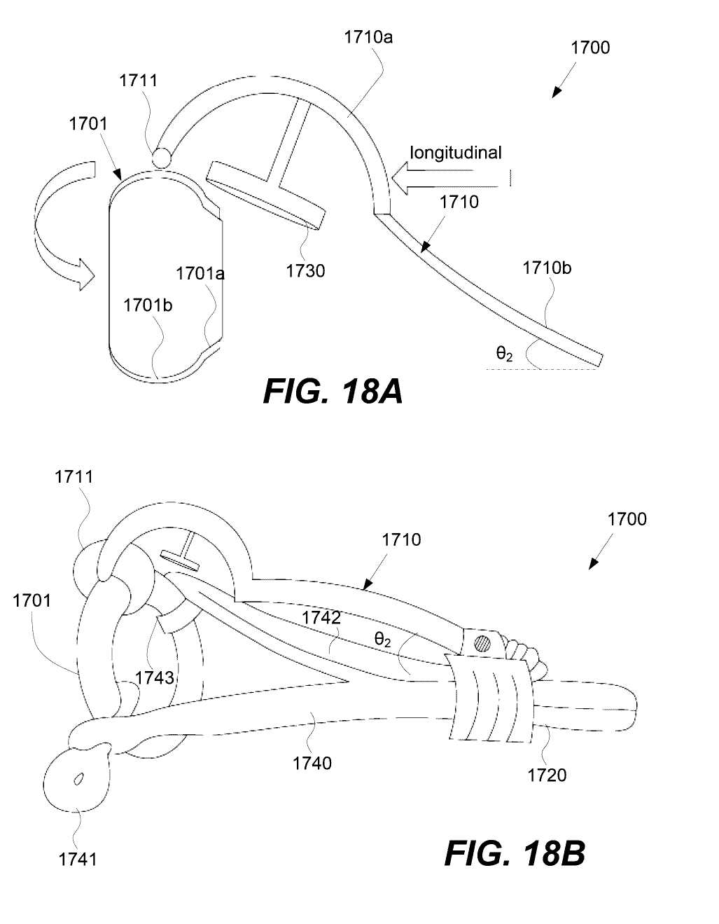

The arm compressor 1720 is connected to the movable arm 1710, the first supporting arm 1740, and a second supporting arm 1742. The arm compressor 1720 can slide along the first supporting arm 1740 and a second supporting arm 1742 while moving the movable arm 1710. Moving the arm compressor 1720 towards the second cutter 1701 flexes the supporting arms 1740 and 1742 inwards exerting a pressure along the circumference of the second cutter 1701 causes the second cutter 1701 to compress. Also, the arm compressor 1720 moves the movable arm 1710 causing the second cutter 1701 to rotated about the third join 1711 by the movable arm 1710 (as shown in FIGS. 18A, 18B, 19A and 19B).

Referring to FIG. 17A, the second cutter 1701 can have a ring like shape with a sharp edge 1701a. The inner portion of the second cutter 1701 can be a curved hollow portion 1701b and the sharp edge 1701a can project from the bottom of the curved hollow portion 1701b. The sharp edge 1701a facilitates cutting of the specimen 130a (in FIG. 4A). The sharp edge 1701a can be angled inwards reducing the opening of the hollow portion 1701b. When the sharp edge 1701a cuts the specimen 130a, the specimen 130a is sucked into the curved hollow portion 1701b of the second cutter 1701 and the specimen 130a expands into the curved hollow portion 1701b. Furthermore, the specimen 130a does not fall when lifted or rotated, as the outward curve of the curved hollow portion 1701b and the inward angle of the sharp edge 1701a prevents the specimen 130a from falling.

The movable arm 1710 has a unitary construction comprising a cup portion 1710a and a shaft portion 1710b. The shaft portion 1710b can be a rod or a plate that can be curved. The cup portion 1710a is arranged in an inverted manner and attached to the shaft portion 1710b. The cup portion 1710a also includes the plunger 1730 projecting downwards from approximately the center of the cup portion 1710. The length of the plunger 1730 is smaller than a radius of the cup portion 1710a to keep the plunger 1730 contained in the cup portion 1710a.

In the first position (i.e., the initial position), the second cutter 1701 is rotated away (outwards) from the cup portion 1710a and positioned such that the sharp edge 1701a points downwards. The sharp edge 1701a is below the level of the shaft portion 1710b to allow cutting of a specimen without touching the arms 1710, 1740 or 1742. The arm compressor 1720 is located at the extreme end of the supporting arms 1740 and 1742 (i.e., a rightmost end in FIG. 17B). The shaft portion 1710b makes an angle θ₁ with respect to the supporting arms 1740 and 1742 (or a horizontal axis).

As the arm compressor 1720 starts moving towards the cup portion 1710a, the second cutter 1710 starts rotating and the angle θ₁ between the movable arm 1710 and the supporting arms 1740 and 1742 starts increasing, as illustrated in FIGS. 18A and 18B.

FIGS. 18A and 18B are a front view and a perspective view, respectively, of the fourth specimen transfer device 1700 in a second position (i.e., an intermediate position). As the arm compressor 1720 moves towards the cup portion 1710a, the movable arms 1710b rotates the second cutter 1701 about the third joint 1711, while also rotating the second cutter 1701 about the first joint 1741 and the second joint 1743. The second cutter 1701, particularly the sharp edge 1701a, rotates (counterclockwise) towards the cup portion 1710a. The movable arm 1710 makes an angle θ₂, greater than the angle θ₁, with the supporting arms 1740 and 1742.

As the arm compressor 1720 moves close to the cup portion 1710a, the angle θ₂ between the movable arm 1710 and the supporting arms 1740 and 1742 starts decreasing, as illustrated in FIGS. 19A and 19B.

FIGS. 19A and 19B are a front view and a perspective view, respectively, of the fourth specimen transfer device 1700 in a third position (i.e., a specimen drop position). As the arm compressor 1720 moves close the cup portion 1710a, the movable arms 1710b rotates the second cutter 1701 into to the cup portion 1710a, fully nesting the second cutter 1701 in the cup portion 1710a. The second cutter 1701 occupies an inverted position with the sharp edge 1701a at the top and pointing into the cup portion 1710a of the movable arm 1710. Furthermore, the plunger 1730 is nested in the hollow portion of the second cutter 1701. In the drop position, the plunger 1730 pushes the specimen 130a (not illustrated) out of the second cutter 1701 which can be dropped into the second specimen plate (not illustrated). The movable arm 1710 makes an angle θ₃, less than angle θ₂, with the supporting arms 1740 and 1742. Thus, the fourth transfer device 1700 can be used to transfer a specimen from the first specimen plate located at a first location to a second specimen plate located at a second location without touching the specimen.

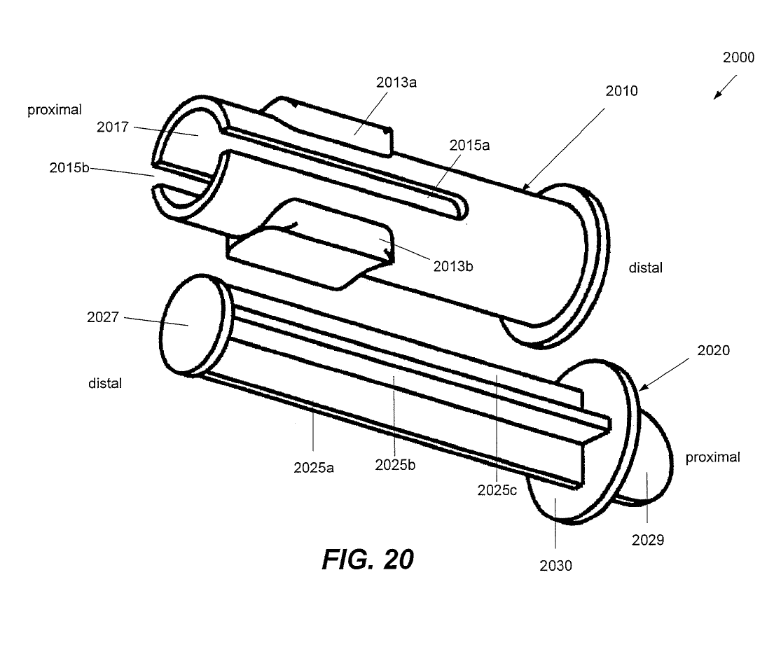

FIG. 20 is a front view of the fifth specimen transfer device in a third position according to certain embodiments of the disclosure. The fifth specimen transfer device 2000 includes a fifth cutter 2010 and a fifth plunger 2020. The fifth cutter 2010 and the fifth plunger 2020 can slide relative to each other when assembled. The fifth cutter 2010 cuts and holds a specimen in a first specimen plate and the fifth plunger 2020 releases the specimen to a second specimen plate.

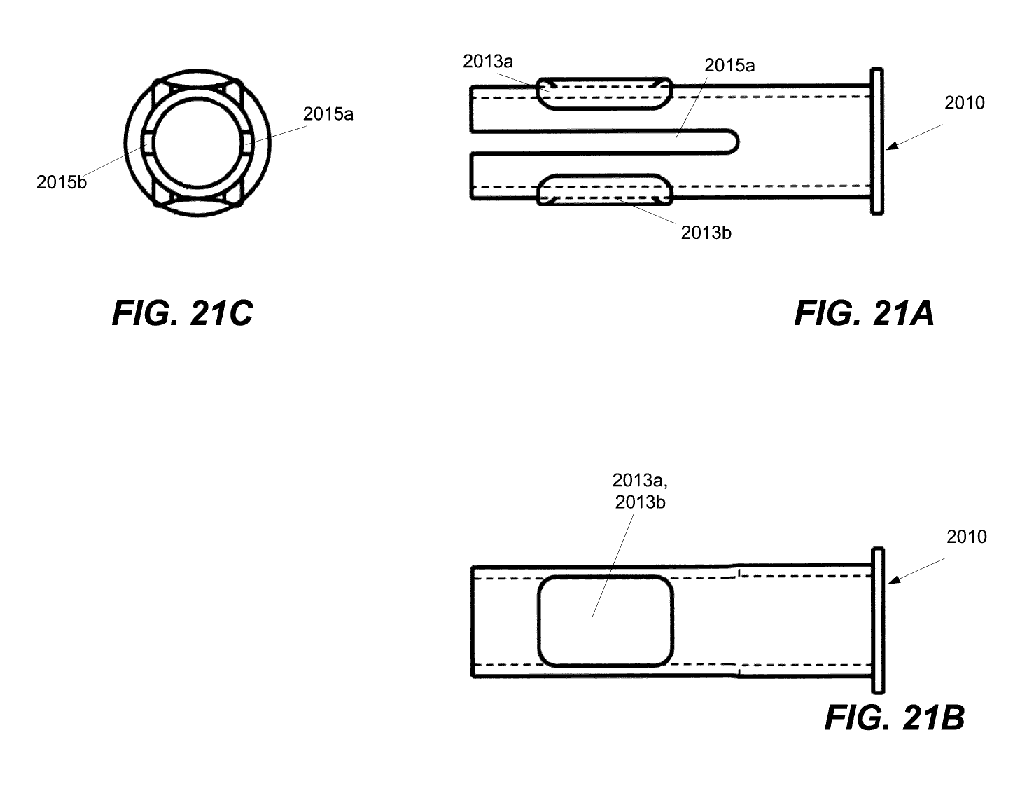

The fifth cutter 2010 is hollow cylindrical in shape. The fifth cutter 2010 includes a two gripping portions 2013a and 2013b and two slots 2015a and 2015b formed along the circumference of the fifth cutter 2010. The slots 2015a and 2015b extend along the length from an open end 2017 (also referred as a proximal end) to a distal end. The slots 2015a and 2015b separated by approximately 1800 from each other. Additional views of the fifth cutter 2010 are illustrated in FIGS. 21A, 21B and 21C.

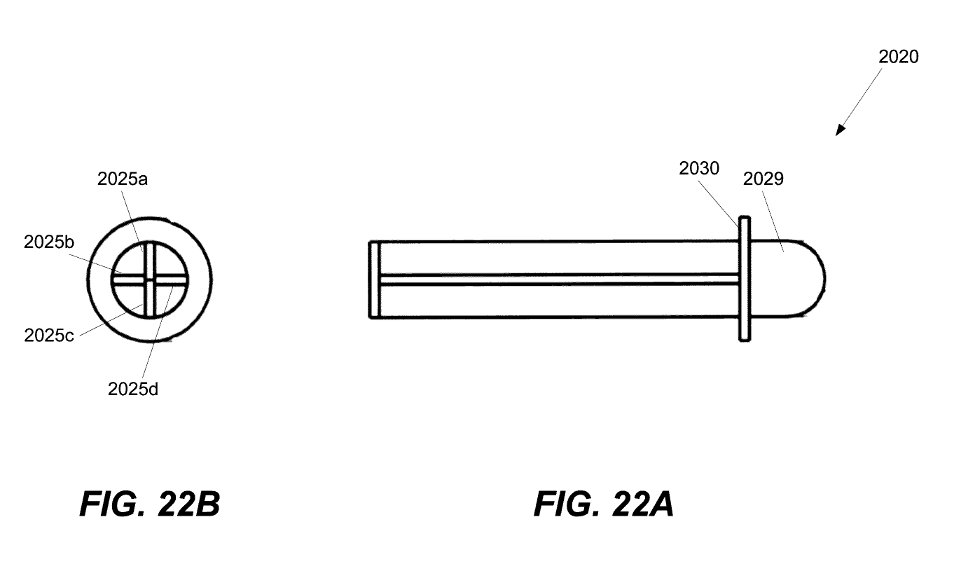

The fifth plunger 2020 is a cylindrical shaft with fins 2025a, 2025b, 2025c, and 2025d projecting along the length. The fins 2025a, 2025b, 2025c, and 2025d make an angle of approximately 90° with respect to each other, refer to FIG. 22B. Referring back to FIG. 20, the fifth plunger 2020 also includes a fifth grip 2029 and a collar 2030 at a proximal end. The distal end of the fifth plunger 2020 has a flat surface 2027. The distal end of fifth plunger 2020 is inserted in the proximal end of the fifth cutter 2010. The fins 2025a and 2025c are inserted in the slots 2015a and 2015b, respectively by gripping the fifth gripper 2029. Additional views of the fifth plunger 2020 are illustrated in FIGS. 22A and 22B.

Referring back to FIG. 20, in operation, a user can hold the fifth cutter 2010 at the gripping portions 2013a and 2013b and cut a specimen from the distal end. To release the specimen, the distal end of fifth plunger 2020 is driven towards the distal end of the fifth cutter 2010 to push out the specimen.

The above-described hardware description is a non-limiting example of corresponding structure for performing the functionality described herein.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

What is claimed is:

1. A specimen transfer apparatus, comprising:
a die cutter having a cylindrical shape and including a hollow interior portion, a spaced gap portion disposed along a cylindrical circumference thereof a cutting edge and a hold attachment, wherein the die cutter is flexible; and
a drive mechanism configured to hold the die cutter via the hold attachment and a drive rod, to constrict the die cutter the drive rod, to release the die cutter from the drive rod, and to rotate the die cutter about an axis of the drive rod from a first position to a second position about the axis of the drive rod.

2. The apparatus according to claim 1, further comprising:
a specimen support surface including a support base, wherein the specimen support surface includes a sensor array disposed in the specimen support surface;
a lift mechanism to drive the specimen support base; and
control processing circuitry,
wherein the control processing circuitry is configured to detect a position of a first pure plate and a position of a second pure plate via the sensor array,
wherein the hold attachment and the drive mechanism are electrically connected to the control processing circuitry, and
wherein the control processing circuitry is further configured to separately control the hold attachment and drive mechanism.

3. The apparatus according to claim 1, wherein the drive mechanism includes at least one drive motor configured to rotate the drive rod, at least one servomotor configured to translate the drive rod, and at least one actuator configured to release and eject the drive rod.

4. The apparatus according to claim 3, wherein the drive rod is connected to the at least one servomotor for translational movement along its axis.

5. The apparatus according to claim 1, wherein the hold attachment of the die cutter includes indents along the outer circumference.

6. The apparatus according to claim 1, wherein the drive rod of the drive mechanism is pivoted for compressing and decompressing the die cutter.

7. The apparatus according to claim 2, wherein the die cutter is configured to cut and grip a sample from the first pure plate.

8. The apparatus according to claim 1, wherein the cutter is a die cutter configured to be removably connected to the drive rod.

9. The apparatus according to claim 1, the drive rod is configured to rotate the die cutter 180 degrees from a position above the first pure plate to a position above the second pure plate.

10. The apparatus according to claim 2, wherein the sensor array is configured to provide an indicator to a user whether the first pure plate and the second pure plate are in their respective pre-set locations.

11. A method for specimen transfer, comprising:
providing an indication that a first pure plate on a specimen support surface is disposed at a first pre-set location;
providing an indication that a second pure plate on the specimen support surface is disposed at a second pre-set location;
determining whether the first pure plate and the second pure plate are aligned on the specimen support surface at a first pre-set location and a second pre-set location, respectively;
raising the specimen support surface to a location proximal a die cutter;
cutting a specimen from the first pure plate via the die cutter;
gripping the specimen via the die cutter and rotating the die cutter 180 degrees from the first pre-set location to the second pre-set location; and
releasing and transferring the cut specimen from the die cutter to the second pure plate.

12. The method according to claim 11, further comprising:
ejecting the used die cutter.

13. The method according to claim 11, wherein the first pure plate includes a nutrient medium containing a microorganism culture.

14. The method according to claim 11, wherein the second plate is a pure plate including a nutrient medium only.

15. The method according to claim 14, wherein the nutrient medium is at least one of Sabouraud's dextrose agar (SabDex), corn meal agar, and potato dextrose agar (PDA).

16. The method according to claim 11, wherein the die cutter is cylindrical in shape and includes a smooth hollow interior portion and a spaced gap portion disposed along its cylindrical circumference.

17. The method according to claim 11, wherein the die cutter is configured to cut and grip the specimen from the first pure plate.

18. A specimen transfer apparatus, comprising:
means for indicating a first pure plate on a specimen support surface is disposed at a first pre-set location;
means for indicating a second pure plate on the specimen support surface is disposed at a second pre-set location;
means for determining whether the first pure plate and the second pure plate are aligned on the specimen support surface at a first pre-set location and a second pre-set location, respectively;
means for raising the specimen support surface to a location proximal a die cutter;
means for cutting a specimen from the first pure plate via the die cutter;
means for gripping the specimen via the die cutter and rotating the die cutter 180 degrees from the first pre-set location to the second pre-set location; and
means for releasing and transferring the cut specimen from the die cutter to the second pure plate.

19. The apparatus according to claim 18, further comprising:
means for ejecting the used die cutter from the apparatus.
